(19) United States
(12) Patent Application Publication
(10) Pub. No.: US 2020/0009068 A1
(43) Pub. Date: Jan. 9, 2020

(54) NANOPARTICLE FORMULATIONS FOR ENHANCED DRUG DELIVERY TO THE BLADDER

(71) Applicant: The Johns Hopkins University, Baltimore, MD (US)

(72) Inventors: Laura Ensign, Towson, MD (US); Justin Hanes, Baltimore, MD (US); Abhijit Date, Baltimore, MD (US); Trinity Bivalacqua, Baltimore, MD (US); Max Kates, Baltimore, MD (US)

(21) Appl. No.: 16/573,688

(22) Filed: Sep. 17, 2019

Related U.S. Application Data
(63) Continuation-in-part of application No. PCT/US2018/022189, filed on Mar. 13, 2018.
(60) Provisional application No. 62/472,935, filed on Mar. 17, 2017.

(51) Int. Cl. A61K 9/51; A61K 31/7068; A61K 31/337; A61K 33/243; A61K 38/21; C07K 16/28; A61K 9/00

(52) U.S. Cl. CPC A61K 9/5146; A61K 31/7068; A61K 31/337; A61K 9/0034; A61K 38/21; C07K 16/2827; A61K 33/243

(57) ABSTRACT

Hypotonic formulations and methods for delivering drugs to bladder, improving drug absorption and retention therein, and minimizing systemic toxicity, are provided. The formulation includes particles formed from the assembly or association between biocompatible polymers with and without low or high grafting density of polyethylene glycol (PEG) and a wide range of drugs. A hypotonic medium or water allows the particles to penetrate and distribute within bladder tissue, where the particles are capable of dissolution to release drugs for absorption and retention. A reduced level of local and systemic toxicity and side effects of the formulation, compared to delivery of drugs in their free form, provides an effective and safe drug delivery platform for treating bladder or associated diseases or disorders.

### CROSS-REFERENCE TO RELATED APPLICATIONS

This application is a continuation-in-part of pending prior International Application No. PCT/US2018/022189, filed Mar. 13, 2018, which claims benefit of and priority to U.S. Provisional Patent Application No. 62/472,935 filed Mar. 17, 2017, which are hereby incorporated herein by reference in their entirety.

### FIELD OF THE INVENTION

The disclosed technology is generally in the field of drug delivery to bladder with increased retention and reduced systemic toxicity.

### BACKGROUND OF THE INVENTION

Bladder cancer is the seventh most common malignancy worldwide, with a five-year global prevalence of 1,319,759 people (Bray F, et al., Int J Cancer, 132(5):1133-1145). Non-muscle invasive bladder cancer (NMIBC) is a most prevalent type bladder cancer which approximately 70% of patients are present with. It is primarily managed by trans-urethral (endoscopic) resection of the bladder tumor followed by intravesical instillation of anti-cancer therapeutics (David K A, et al., Cancer, 115(7):1435-1447 (2009)). For more than 35 years, bacillus Calmette-Guerin (BCG), a live attenuated strain of Mycobacterium bovis, has been the mainstay for intravesical treatment of NMIBC to reduce risks of tumor progression. Nevertheless, more than 50% of patients are intolerant to BCG or still subject to recurrence or progression of bladder cancer, and no standard alternative intravesical therapy is available (Chang S S, et al., J Urol 196(June):1-9 (2016)).

Bladder epithelium has specific structures and functions that are unlike the colon, the vagina, and other mucosal surfaces that absorb water across the epithelium to re-establish osmotic equilibrium. The bladder epithelium forms fluid-filled vesicles that can reversibly fuse with the bladder epithelium. It maintains osmotic equilibrium using mechanisms that do not involve water transfer across the epithelial surface. The bladder epithelium is exposed to urine with a composition widely different from that of plasma (isotonic).

Ineffective retention of treatment modalities in all layers of the bladder is a major factor that limits the success of therapies in benign and malignant disease of the bladder. For example, current clinical practice for intravesical NMIBC therapies includes catheterization of the patient to administer therapeutic agent into the bladder followed by temporarily sealing the catheter to facilitate absorption and retention of the therapeutic agent in the bladder. During this time, urine production dilutes the therapeutic agent in the bladder (GuhaSarkar S, et al., J Control Release, 148(2):147-159 (2010); Douglass L, et al., Bl Cancer, 2(3):285-292 (2016)). Moreover, the majority of the drug in the bladder is subsequently lost by urination following catheterization. Therefore, prolonging the residence time of therapeutics in the bladder is challenging and of great interest for intravesical therapy.

Mitigating the toxicity of chemotherapeutics in the treatment of bladder cancer is also challenging. For example, cisplatin (cis-diamminedichloridoplatinum (II), CDDP) is an alkylating antineoplastic agent that interferes with DNA replication, thereby killing fast proliferating cells. One mechanism of action involves crosslinking DNA in a process where cisplatin has one of the two chloride ligands displaced by water following in vivo administration to give rise to an aqua complex whose aqua ligand is easily displaced by an N-heterocyclic base (e.g., guanine) on DNA and whose other chloride ligand is further displaced by another N-heterocyclic base on DNA, forming a crosslink damage in the DNA. Although systemic administration of cisplatin-based chemotherapy has shown superior efficacy to other chemotherapeutic for patients with locally advanced or metastatic bladder cancer (von der Masse H, et al., J Clin Oncol, 18(17):3068-77 (2000)), hypersensitivity reactions, which can occur with small quantities of circulating platinum, are present in 5-20% of the cases even during systemic CDDP therapy (Makrilia N, et al., Met Based Drugs, 2010:207084 (2010)). Alternatively with intravesical administration, a clinical trial for treatment of superficial bladder cancer has found, of the 50 patients who received intravesically administered CDDP for more than 4 months, 3 (6%) stopped treatment early due to chemical cystitis, and 7 (14%) had an anaphylactic reaction due to systemic drug absorption resulting in hypotensive shock (Denis L, Lancet, 1(8338):1378-9 (1983); trial conducted by the European Organization for Research on the Treatment of Cancer EORTC 30782 in 1981). As a result, the CDDP arm of the study was suspended, and the high rate of anaphylaxis and incidences of local toxicity precluded further exploration of intravesical CDDP for the treatment of NMIBC. Hypersensitivity associated with intravesical administration of CDDP was likely due to translocation of CDDP into the systemic circulation.

Although nanotechnology approaches have been studied to improve the efficacy and/or reduce adverse effects associated with various chemotherapeutics by controlling drug release and modulating bio-distribution (Hare J l, et al., Adv Drug Deliv Rev, 108:25-38 (2017)), few studies of NPs for intravesical therapy to date have looked at or been designed with consideration to bladder retention in vivo (Martin, D T, et al., Nanomedicine Nanotechnology, Bio Med, 9(8):1124-1134 (2013); Kang M, et al., Cancer Res, 72(19):5069-5079 (2012); Mugabe C, et al., Clin Cancer Res, 17(9):2788-2798; Mugabe C, et al., Biomaterials, 33(2):692-703 (2012)). Pre-clinical animal experiments and phase I human trials in solid tumors show systemic nanoparticle therapies may reduce the toxic side effects associated with CDDP while maintaining or improving efficacy (Mizumura Y, et al., Japanese J Cancer Res 92(3):328-36 (2001); Uchino H, et al., Br J Cancer 93(6):678-87 (2005); Plummer R, et al., Br J Cancer 104(4):593-598 (2011); Paraskar A S, et al., PNAS, 107(28):12435-12440 (2010); Sengupta P, et al., PNAS, 109(28):11294-11299 (2012); Oberoi H S, et al, Adv Drug Deliv Rev 65(13-14):1667-1685 (2013); Matsumura Y, Jpn J Clin Oncol 44(6):515-525 (2014); Nishiyama N, et al., Cancer Sci 107(7):867-874 (2016); McKiernan J M, et al., J Urol 192(6):1633-1638 (2014)). However, the formulations are generally directed to cancers in other tissues and the studies do not look at drug retention in the bladder.

Design criteria for drug uptake, retention, and efficacy in the bladder may differ from those for other tissues considering the structural and functional differences in different organs. One problem is that bladder undergoes voiding and refill processes from urination and metabolism, which often leads to reversible nanoparticle uptake in which a nanoparticle vehicle may be quickly voided from bladder tissue before drug agents are released.

Another problem is that the bladder does not have a soluble mucin gel layer coating the epithelium (N'Dow J, et al., J Urol 173(6):2025-2031 (2005)), therefore previous studies which show pegylation of nanoparticles may increase mucosal tissue distribution and delivery to mucosal surfaces like the gastrointestinal tract, airways, and female reproductive tract (Suk J S, et al., Adv Drug Deliv Rev 99:28-51 (2016); U.S. Patent Application Publication No. US20140329913; U.S. Pat. No. 9,415,020) are not applicable.

Therefore, it is an object of the present invention to provide formulations for effective, rapid, uniform, and deep coverage and safe delivery of a wide range of drugs to bladder tissue with minimal systemic toxicity.

It is also another object of the present invention to provide a method of treating bladder diseases and disorders with formulations that are effective and safe.

### SUMMARY OF THE INVENTION

A formulation is provided to effectively deliver a wide range of agents to bladder with reduced systemic toxicity. The formulation generally includes nano- or micro-particles prepared from a polymer and a therapeutic, prophylactic, diagnostic or nutraceutical agent in water or in an excipient hypotonic for urothelial epithelium. Exemplary particles are generally in a form of, but not limited to, micelles, colloids, liposomes, vesicles, nanodroplets, nano-structured hydrogel, nanocrystals, or nanosuspension. The particles are generally formed via the assembly or association (non-covalently or covalently) between the polymer and the agent and are capable of dissolution or disassembly to release the agent in the bladder tissue. The agents retain their bioactivity even after assembly or association with the polymer.

In preferred embodiments, the particles are delivered in water or a medium containing excipients that are hypotonic for a urothelial epithelium (e.g., renal pelvis, ureters, bladder, or urethra epithelium) to enhance the penetration and distribution of particles in the bladder tissue. Generally the medium has an osmolality less than 400 mOsm/kg (urine osmolality generally varies between 400 and 900 mOsm/kg) or less than 220 mOsm/kg. The water or hypotonic medium of the formulation generally allows the particles to penetrate into the bladder tissue to a greater extent than an isotonic saline solution (e.g., phosphate-buffered saline). The particles in the water or hypotonic medium uniformly distribute in the bladder tissue. The agent released from the particle is generally retained in the bladder tissue to a greater extent than is the agent delivered in its free soluble form. In some embodiments, the particles do not contain a polyalkylene polymer coating, or the polymers forming the particles do not contain polyethylene glycol or contain polyethylene glycol to a limited extent (e.g., less than 70%, 50%, 30%, or 15% in weight of the polymer). In some embodiments, the particles not containing polyethylene glycol (PEG) or containing it to a limited extent, show an improved penetration and distribution within the bladder tissue compared to ones containing a greater amount of PEG.

In some embodiments where metal-based therapeutics are delivered, e.g., delivering platinum-based agent, particles are formed with polymers capable of forming polymer→metal (e.g., O→Pt) coordination, generally via ligand substitution reaction, to reversibly bond with the agent, such that the particles are capable of dissolution in bladder tissue to release the agent. In some embodiments, the formulation contains nanoparticles formed from the complexation between cisplatin and poly-aspartic acid (PAA), e.g., via ligand substitution reaction at the platinum atom of cisplatin. In these particles, the aspartic acid functional groups on the polymer are ligands to and bonded with the platinum of cisplatin. The PAA may be conjugated to PEG, although a low content of PEG generally results in an improved absorption and retention of the particles and delivered agents in the bladder tissue.

In other embodiments, the formulation contains nanosuspensions formed between an agent and block copolymers including poloxamers such as PLURONIC® and KOLLIPHOR® polymers. In these nanosuspensions, the agent is generally dispersed and encapsulated.

Unlike intravesical administration of drug agents in their free or soluble form, intravesical administration of the particles generally results in limited, and in some embodiments, undetectable systemic levels of the agents, effectively reducing the toxicity and side effects of these agents. These formulations also do not lead to bladder tissue hyperplasia or increase of bladder weights, which are present with repeated intravesical administration of drug agents in their free form. In some embodiments where a subject contains non-muscle invasive bladder cancer, treatment with the hypotonic formulations containing the dissolvable particles to deliver anti-cancer agents improves chemoprophylactic activity and enhances immune response of the subject, compared to treatment using the anti-cancer agent in its free form, especially in a delivery route of intravesical instillation.

### BRIEF DESCRIPTION OF THE DRAWINGS

FIG. 1 is a schematic showing the formation of complexed nanoparticles between cisplatin (CDDP) and poly-aspartic acid.

FIG. 2 is a line graph showing the percent of cell viability of RT4 cells (human bladder transitional cell papilloma cell) over the amounts of platinum (μg/mL) from CDDP (denoted as a diamond-shaped symbol), carboplatin (denoted as a triangle symbol), or nanoparticles formed from the complexation between CDDP and poly-aspartic acid (PAA-CDDP NP; denoted as a circle symbol).

FIG. 3 is a line graph showing the percent of cell viability of 5637 cells (human bladder grade II carcinoma cell) over the amounts of platinum (μg/mL) from CDDP (denoted as a diamond-shaped symbol), PAA-CDDP NP (denoted as a square symbol), and nanoparticles formed from the complexation between densely pegylated poly-aspartic acid and CDDP (PEG_{high}-PAA-CDDP NP; denoted as a circle symbol).

FIG. 4 is a line graph showing the percent of cell viability of J82 cells (human bladder transitional cell carcinoma) over the amounts of platinum (μg/mL) from CDDP (denoted as a diamond-shaped symbol), PAA-CDDP NP (denoted as a square symbol), and PEG_{high}-PAA-CDDP NP (denoted as a circle symbol).

FIG. 5 is a bar graph showing the amount of cisplatin in mouse plasma (μg/mL) 1 hour following intravesical administration of CDDP solution or PAA-CDDP NPs in mice.

FIG. 6 is a bar graph showing the bladder weights (mg) of mice following three weekly intravesical administrations in mice of saline (sham control, untreated), CDDP solution, or PAA-CDDP NPs.

FIG. 7 is a bar graph showing the amount of cisplatin in mouse bladder at 1 hour, 4 hours, and 24 hours following intravesical administration in mice of CDDP solution, PAA-CDDP NPs, PEG_{low}-PAA-CDDP NP, or PEG_{high}-PAA-CDDP NP.

FIG. 8 is a bar graph showing the amount of cisplatin in rat bladder at 1 hour and 4 hours following intravesical administration in rats of CDDP solution, PAA-CDDP NPs, PEG_{low}-PAA-CDDP NP, or PEG_{high}-PAA-CDDP NP.

FIG. 9 is a bar graph showing the percentage of tumor in the in situ stage and that in the invasive (T1) stage in rat bladder when administered intravesically with saline (untreated), CDDP in solution, or PAA-CDDP NPs.

FIGS. 10-13 are bar graphs showing the numbers of Ki67 positive cells (FIG. 10), CD8 positive cells (FIG. 11), CD3 positive cells (FIG. 12), and Foxp3 positive cells (FIG. 13) from high power field (HPF) microscopic images of rat bladder immunohistochemistry specimens for bladder carcinogenesis-induced rats administered intravesically with saline (untreated), CDDP in solution, or PAA-CDDP NPs.

FIG. 14 is a graph showing the particle sizes (nm) and polydispersity indices of docetaxel nanocrystals/nanosuspensions formed with PLURONIC® F127, PLURONIC® F68, KOLLIPHOR® HS 15, or KOLLIPHOR® TPGS.

FIG. 15 is a graph showing the particle sizes (nm) and polydispersity indices of docetaxel nanocrystals/nanosuspensions over time (hours).

FIGS. 16-18 are line graphs showing the percent of cell viability of RT4 cells (FIG. 16), 5637 cells (FIG. 17), and J82 cells (FIG. 18) over the amounts of docetaxel (DTX; ng/mL) from DTX in solution (denoted as circular symbol) or DTX nanosuspension (DTX NS; denoted as square symbols).

FIG. 19 is a bar graph showing the concentrations of docetaxel in bladder tissue (ng/g) at 1 hour following intravesical administration in rats of taxotere in water, taxotere in saline, DTX NS in water, or DTX NS in saline.

FIG. 20 is a bar graph showing the concentrations of docetaxel in bladder tissue (ng/g) at 1 hour, 2 hours, and 4 hours following intravesical administration in rats of taxotere in water or of DTX NS in water.

FIG. 21 is a bar graph showing the concentrations of docetaxel in plasma (ng/mL) at 1 hour following intravesical administration in rats of taxotere in water or of DTX NS in water.

FIG. 22 is a bar graph showing the percentage of tumor in the invasive stage, in situ stage, in dysplasia stage, or no cancer in rat bladder when administered intravesically with saline (untreated), taxotere, or DTX NS.

FIG. 23 is a bar graph showing the numbers of Ki67 positive cells from high power field (HPF) microscopic images of rat bladder immunohistochemistry specimens for bladder carcinogenesis-induced rats administered intravesically with saline (untreated), taxotere, or DTX NS.

### DETAILED DESCRIPTION OF THE INVENTION

### I. Definitions

The term “nanoparticle,” as used herein, generally refers to a structure of any shape having a diameter from about 1 nm up to, but not including, about 1 micron, more preferably from about 5 nm to about 500 nm. Nanoparticles having a spherical shape are generally referred to as “nanospheres”. Non-limiting examples of nanoparticles include soft nanoparticles, e.g., micelles, colloids, liposomes, vesicles, nanodroplets nano-structured hydrogel, nanocrystals, and nanosuspension. Soft nanoparticles generally dissolve or dissemble to release agents.

The term “nanocrystal,” as used herein refers to a material particle having at least one dimension smaller than 100 nanometres and composed of atoms in either a single- or poly-crystalline arrangement.

The term “nanosuspension,” as used herein refers to a submicron colloidal, dispersion of drug particles.

The term “IC₅₀”, as used herein, refers to a concentration of an inhibitor (or tested agent) where the response (or binding) is reduced by half.

The term “coordination complex,” as used herein, refers to coordination compounds containing ions or molecules that are linked, or coordinated, to a transition metal (e.g., Pt, Ni, Pd, Rh, Ir, Au, Zn, and Cu).

The terms “ligand” and “metal coordination ligand” herein refer to ions or molecules that can bind to transition-metal ions to form complexes. The number of ligands bound to the transition metal ion is called the coordination number. Any ion or molecule with a pair of nonbonding electrons can be a ligand. Many ligands are described as monodentate (e.g., “one-toothed”) because they “bite” the metal in only one place. Monodentate ligands refer to ligands that have only one donor atom attached to the metal center. Bidentate ligands refer to ligands that have two donor atoms attached to the same metal center. Tridentate ligands refer to ligands that have three donor atoms attached to the same metal center. Tetradentate ligands refer to ligands that have four donor atoms attached to the same metal center. The term “chelate” means “claw” from its Greek stem and is used to describe ligands that can grab the metal in two or more places.

The term “polymer” refers to a chemical entity with a plurality of repeating units generally bonded covalently. In some forms, a polymer has a molecular weight greater than 500 or 1,000, or more. Non-limiting exemplary polymers include polyamino acids, naturally-occurring, and synthetic chemical compounds.

The term “pharmaceutically acceptable,” as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration.

The terms “biocompatible” and “biologically compatible,” as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

The term “transitional epithelium,” as used herein refers to a type of tissue including multiple layers (e.g., basal, intermediate, and superficial) of epithelial cells which can contract and expand, which functions in the transition of degree of distension. This tissue structure type is found in urothelium, including that of the renal pelvis, urinary bladder, the ureters, the superior urethra, and the prostatic and ejaculatory ducts of the prostate.

The term “molecular weight,” as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

The term “hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

The term “hydrophobic,” as used herein, refers to the property of lacking affinity for or repelling water. For example, the more hydrophobic a polymer (or polymer segment), the more that polymer (or polymer segment) tends to not dissolve in, not mix with, or not be wetted by water.

The term “POLOXAMER” as used herein is a trademark referring to nonionic triblock copolymers composed of a central hydrophobic chain of polyoxypropylene (poly(propylene oxide)) flanked by two hydrophilic chains of polyoxyethylene (poly(ethylene oxide)).

The term “surfactant” as used herein refers to an agent that lowers the surface tension of a liquid.

The term “therapeutic agent” refers to an agent that can be administered to prevent or treat a disease or disorder. Therapeutic agents can be a nucleic acid, a nucleic acid analog, a small molecule, a peptidomimetic, a protein, peptide, carbohydrate or sugar, lipid, or surfactant, or a combination thereof.

The term “treating” or “preventing” a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain.

The term “targeting moiety” as used herein refers to a moiety that localizes to or away from a specific locale. The moiety may be, for example, a protein, nucleic acid, nucleic acid analog, carbohydrate, or small molecule. The entity may be, for example, a therapeutic compound such as a small molecule, or a diagnostic entity such as a detectable label. The locale may be a tissue, a particular cell type, or a subcellular compartment.

The term “therapeutically effective amount” refers to an amount of the therapeutic agent that, when incorporated into and/or onto particles described herein, produces some desired effect at a reasonable benefit/risk ratio applicable to any treatment. The effective amount may vary depending on such factors as the disease or condition being treated, the particular formulation being administered, the size of the subject, or the severity of the disease or condition.

The terms “incorporated” and “encapsulated” refers to incorporating, formulating, or otherwise including an agent into and/or onto a composition, regardless of the manner by which the agent or other material is incorporated.

Conventional use of the term “isotonic” refers to fluids that do not cause cells to swell or shrink, which typically occurs when the total solute concentrations (osmolality) is equal to that of the blood (˜300 mOsm/kg) although values differ for different tissues. Isotonic is defined herein as a formulation that does not cause water to enter or leave the lumen or be driven osmotically through the epithelium. Hypotonic is defined herein to refer to formulations that cause water to flow inward, toward the epithelium from the mucosal surface, and hypertonic formulations are defined as those that cause water to flow outward, toward the mucus-coated surface.

### II. Composition

Formulations for effective delivery into the bladder include particles delivering a wide range of agents, and are in some embodiment in a hypotonic medium or water to enhance the penetration into the bladder tissue. These particles are generally formed via the assembly or association between a polymer and agent, such that the particles are capable of dissolution or disassembly to release the agent in the bladder tissue, leading to an improved absorption and retention of agents in bladder. These particles may be nanosized or have a size in the micrometer range. They are generally in the form of micelles, colloids, liposomes, vesicles, nanodroplets nano-structured hydrogel, nanocrystals, or nanosuspension.

1. Polymer

Biocompatible polymers are generally used to prepare the particles. In one embodiment, the biocompatible polymer(s) is biodegradable or bioabsorbable. In another embodiment, the polymer is non-degradable. In other embodiments, the particles are a mixture of degradable and non-degradable particles.

In some embodiments where metal-based agents are delivered, e.g., delivering platinum-based chemotherapeutics, polymer is generally capable of forming polymer→metal (e.g., O→Pt) coordination, generally via ligand substitution reaction, to reversibly bond with the agent. These polymers include, but are not limited to, polyamino acids such as poly(aspartic acid) (PAA) and poly(glutamic acid); polymers containing polymaleic acid or polymaleic anhydride; polymers with an attached cholesterol.

Exemplary polymers to form “soft” nanoparticles (e.g., capable of dissolution to release agent) include, but are not limited to, other polyamino acids; cyclodextrin-containing polymers, in particular cationic cyclodextrin-containing polymers, such as those described in U.S. Pat. No. 6,509,323; polymers prepared from lactones such as poly(caprolactone) (PCL); polyhydroxy acids and copolymers thereof such as poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), and blends thereof, polyalkyl cyanoacralate, polyurethanes, poly(valeric acid), and poly-L-glutamic acid; hydroxypropyl methacrylate (HPMA); polyanhydrides; other polyesters; polyorthoesters; poly(ester amides); polyamides; poly(ester ethers); polycarbonates; polyalkylenes such as polyethylene and polypropylene; polyalkylene glycols such as poly(ethylene glycol) (PEG) and polyalkylene oxides (PEO), and block copolymers thereof such as polyoxyalkylene oxide (“PLURONICS®” or block copolymers containing PEG where PEG has a molecular weight of any values within the range of 300 Daltons to 1 MDa); polyalkylene terephthalates such as poly(ethylene terephthalate); ethylene vinyl acetate polymer (EVA); polyvinyl alcohols (PVA); polyvinyl ethers; polyvinyl esters such as poly(vinyl acetate); polyvinyl halides such as poly(vinyl chloride) (PVC), polyvinylpyrrolidone; polysiloxanes; polystyrene (PS); and celluloses including alkyl celluloses, hydroxyalkyl celluloses, cellulose ethers, cellulose esters, nitro celluloses, hydroxypropylcellulose, and carboxymethylcellulose; polymers of acrylic acids including poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate) (jointly referred to herein as “polyacrylic acids”); polydioxanone and its copolymers; polyhydroxyalkanoates; polypropylene fumarate; polyoxymethylene; poloxamers; poly(butyric acid); trimethylene carbonate; and polyphosphazenes.

Examples of preferred natural polymers include proteins such as albumin, collagen, gelatin and prolamines, for example, zein, and polysaccharides such as alginate.

Copolymers of the above, such as random, block, or graft copolymers, or blends of the polymers listed above can also be used.

Functional groups on the polymer can be capped to alter the properties of the polymer and/or modify (e.g., decrease or increase) the reactivity of the functional group. For example, the carboxyl termini of carboxylic acid contain polymers, such as lactide- and glycolide-containing polymers, may optionally be capped, e.g., by esterification, and the hydroxyl termini may optionally be capped, e.g. by etherification or esterification.

The weight average molecular weight can vary for a given polymer but is generally from about 1000 Daltons to 1,000,000 Daltons, 1000 Daltons to 500,000 Dalton, 1000 Daltons to 250,000 Daltons, 1000 Daltons to 100,000 Daltons, 5,000 Daltons to 100,000 Daltons, 5,000 Daltons to 75,000 Daltons, 5,000 Daltons to 50,000 Daltons, or 5,000 Daltons to 25,000 Daltons.

In some embodiments, the particles may be used as nanoparticle gene carriers. In these embodiments, the particles can be formed of one or more polycationic polymers which complex with one or more nucleic acids which are negatively charged. The cationic polymer can be any synthetic or natural polymer bearing at least two positive charges per molecule and having sufficient charge density and molecular size to bind to nucleic acid under physiological conditions (i.e., pH and salt conditions encountered within the body or within cells). In certain embodiments, the polycationic polymer contains one or more amine residues.

In some embodiments, the particles are formed with surfactants. Examples of surfactants include, but are not limited to, L-α-phosphatidylcholine (PC), 1,2-dipalmitoylphosphatidycholine (DPPC), oleic acid, sorbitan trioleate, sorbitan mono-oleate, sorbitan monolaurate, polyoxyethylene (20) sorbitan monolaurate, polyoxyethylene (20) sorbitan monooleate, natural lecithin, oleyl polyoxyethylene (2) ether, stearyl polyoxyethylene (2) ether, lauryl polyoxyethylene (4) ether, block copolymers of oxyethylene and oxypropylene, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol 400, cetyl pyridinium chloride, benzalkonium chloride, olive oil, glyceryl monolaurate, corn oil, cotton seed oil, and sunflower seed oil, lecithin, oleic acid, and sorbitan trioleate.

In some embodiments where polyalkylene glycol (e.g., PEG) is used as in the polymer to form the particles for delivery to bladder, PEG surface density may be controlled by varying the amount of PEG in the polymer composition or by mixing a blend of pegylated polymer component and non-pegylated polymer component. The density of PEG or polyalkylene glycol on the surface of formed particles may be evaluated using several techniques. For example, nuclear magnetic resonance (NMR), both qualitatively and quantitatively (PEG peak typically observed ˜3.65 ppm). When particles are dispersed within the NMR solvent D₂O, only the surface PEG, not the PEG embedded within the core, can be directly detected by NMR. Therefore, NMR provides a means for directly measure the surface density of PEG. In some forms, delivery to bladder tissue shows an improved drug absorption and retention when lower amount or no PEG is used. In these forms, PEG density is below approximately 20, 10, or five PEG chains/100 nm², or the mass of PEG in the particle excluding the active agent is less than 70%, 50%, 30%, 25%, or 10%.

In some embodiments, the particles possess a ζ-potential of between about 20 mV and about −20 mV, preferably between about 10 mV and about −10 mV, more preferably between about 2 mV and about −2 mV.

2. Therapeutic, Prophylactic, and Diagnostic Agents

A wide range of agents may be included in the particles to be delivered to bladder. These may be proteins or peptides, sugars or carbohydrate, nucleic acids or oligonucleotides, lipids, small molecules, or combinations thereof. In some embodiments, the particles have encapsulated therein, dispersed therein, and/or covalently or non-covalently associate with the surface one or more agents.

a. Therapeutic Agents

Exemplary classes of therapeutic agents include, but are not limited to, analgesics, anti-inflammatory drugs, anti-proliferatives such as anti-cancer agents like gemcitabine alone or in combinations, such as the combination of cabazitaxel, cisplatin and gemcitabine, anti-infectious agents such as antibacterial agents and antifungal agents (e.g., levofloxacin, CIPRO, ciprofloxacin, cephalexin, ZOTRIM, BACTRIM, MACROBID, nitrofurantoin, fosfomycin, methenamine hippurate, TRIMPEX, PROLOPRIM, trimethroprim, nalidixic acid, and phenazopyridine), antihistamines, corticosteroids, dopaminergics, and muscle relaxants.

In some embodiments, the agent is one or more nucleic acids. The nucleic acid can alter, correct, or replace an endogenous nucleic acid sequence. The nucleic acid is used to treat cancers, correct defects in genes in one or more bladder or urothelial diseases or disorders.

Gene therapy is a technique for correcting defective genes responsible for disease development. Researchers may use one of several approaches for correcting faulty genes: A normal gene may be inserted into a nonspecific location within the genome to replace a nonfunctional gene. An abnormal gene can be swapped for a normal gene through homologous recombination. The abnormal gene can be repaired through selective reverse mutation, which returns the gene to its normal function. The regulation (the degree to which a gene is turned on or off) of a particular gene can be altered.

The nucleic acid can be a DNA, RNA, a chemically modified nucleic acid, or combinations thereof. For example, methods for increasing stability of nucleic acid half-life and resistance to enzymatic cleavage are known in the art, and can include one or more modifications or substitutions to the nucleobases, sugars, or linkages of the polynucleotide. The nucleic acid can be custom synthesized to contain properties that are tailored to fit a desired use. Common modifications include, but are not limited to use of locked nucleic acids (LNAs), unlocked nucleic acids (DNAs), morpholinos, peptide nucleic acids (PNA), phosphorothioate linkages, phosphonoacetate linkages, propyne analogs, 2′-O-methyl RNA, 5-Me-dC, 2′-5′ linked phosphodiester linage, Chimeric Linkages (Mixed phosphorothioate and phosphodiester linkages and modifications), conjugation with lipid and peptides, and combinations thereof.

In some embodiments, the nucleic acid includes internucleotide linkage modifications such as phosphate analogs having achiral and uncharged intersubunit linkages (e.g., Sterchak, E. P. et al., Organic Chem., 52:4202, (1987)), or uncharged morpholino-based polymers having achiral intersubunit linkages (see, e.g., U.S. Pat. No. 5,034,506). Some internucleotide linkage analogs include morpholidate, acetal, and polyamide-linked heterocycles. Other backbone and linkage modifications include, but are not limited to, phosphorothioates, peptide nucleic acids, tricyclo-DNA, decoy oligonucleotide, ribozymes, spiegelmers (containing L nucleic acids, an apatamer with high binding affinity), or CpG oligomers.

Phosphorothioates (or S-oligos) are a variant of normal DNA in which one of the nonbridging oxygens is replaced by a sulfur. The sulfurization of the internucleotide bond dramatically reduces the action of endo- and exonucleases including 5′ to 3′ and 3′ to 5′ DNA POL 1 exonuclease, nucleases S1 and P1, RNases, serum nucleases and snake venom phosphodiesterase. In addition, the potential for crossing the lipid bilayer increases. Because of these important improvements, phosphorothioates have found increasing application in cell regulation.

Peptide nucleic acids (PNA) are molecules in which the phosphate backbone of oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that is similar to oligonucleotides, but are achiral and neutrally charged molecules. Peptide nucleic acids are typically comprised of peptide nucleic acid monomers. The heterocyclic bases can be any of the standard bases (uracil, thymine, cytosine, adenine and guanine) or any of the modified heterocyclic bases described below. A PNA can also have one or more peptide or amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers). Methods for the chemical assembly of PNAs are well known.

In some embodiments, the nucleic acid includes one or more chemically-modified heterocyclic. In some embodiments the nucleic acid includes one or more sugar moiety modifications, including, but are not limited to, 2′-O-aminoethoxy, 2′-O-amonioethyl (2′-OAE), 2′-O-methoxy, 2′-O-methyl, 2-guanidoethyl (2′-OGE), 2′-0,4′-C-methylene (LNA), 2′-O-(methoxyethyl) (2′-OME) and 2′-O—(N-(methyl)acetamido) (2′-OMA).

Methods of gene therapy typically rely on the introduction into the cell of a nucleic acid molecule that alters the genotype of the cell. Introduction of the nucleic acid molecule can correct, replace, or otherwise alters the endogenous gene via genetic recombination. Methods can include introduction of an entire replacement copy of a defective gene, a heterologous gene, or a small nucleic acid molecule such as an oligonucleotide. This approach typically requires delivery systems to introduce the replacement gene into the cell, such as genetically engineered viral vectors.

Methods to construct expression vectors containing genetic sequences and appropriate transcriptional and translational control elements are well known in the art. These methods include in vitro recombinant DNA techniques, synthetic techniques, and in vivo genetic recombination. Expression vectors generally contain regulatory sequences necessary elements for the translation and/or transcription of the inserted coding sequence. For example, the coding sequence is preferably operably linked to a promoter and/or enhancer to help control the expression of the desired gene product. Promoters used in biotechnology are of different types according to the intended type of control of gene expression. They can be generally divided into constitutive promoters, tissue-specific or development-stage-specific promoters, inducible promoters, and synthetic promoters.

Viral vectors include adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA.

Gene targeting via target recombination, such as homologous recombination (HR), is another strategy for gene correction. Gene correction at a target locus can be mediated by donor DNA fragments homologous to the target gene (Hu, et al., Mol. Biotech., 29:197-210 (2005); Olsen, et al., J. Gene Med., 7:1534-1544 (2005)). One method of targeted recombination includes the use of triplex-forming oligonucleotides (TFOs) which bind as third strands to homopurine/homopyrimidine sites in duplex DNA in a sequence-specific manner. Triplex forming oigonucleotides can interact with either double-stranded or single-stranded nucleic acids. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a Kd less than 10-6, 10-8, 10-10, or 10-12. Methods for targeted gene therapy using triplex-forming oligonucleotides (TFO's) and peptide nucleic acids (PNAs) are described in U.S. Published Application No. 20070219122 and their use for treating infectious diseases such as HIV are described in U.S. Published Application No. 2008050920. The triplex-forming molecules can also be tail clamp peptide nucleic acids (tcPNAs), such as those described in U.S. Published Application No. 2011/0262406.

Double duplex-forming molecules, such as a pair of pseudocomplementary oligonucleotides, can also induce recombination with a donor oligonucleotide at a chromosomal site. Use of pseudocomplementary oligonucleotides in targeted gene therapy is described in U.S. Published Application No. 2011/0262406.

b. Diagnostic Agents

Exemplary diagnostic materials include paramagnetic molecules, fluorescent compounds, magnetic molecules, and radionuclides. Suitable diagnostic agents include, but are not limited to, x-ray imaging agents and contrast media. Radionuclides also can be used as imaging agents. Examples of other suitable contrast agents include gases or gas emitting compounds, which are radioopaque. Nanoparticles can further include agents useful for determining the location of administered particles. Agents useful for this purpose include fluorescent tags, radionuclides and contrast agents.

These agents can also be used prophylactically.

3. Tonicity

Tonicity is the ‘effective osmolality’ and is equal to the sum of the concentrations of the solutes which have the capacity to exert an osmotic force across the membrane. A number of different materials can be used to adjust tonicity. For example, the USP 29-NF 24 lists five excipients classified as “tonicity” agents, including dextrose, glycerin; potassium chloride; mannitol; and sodium chloride See, for example, United States Pharmacopeial Convention, Inc. United States Pharmacopeia 29-National Formulary 24. Rockville Md.: U.S. Pharmacopeial Convention, Inc.; 2005: 3261; Day, A. Dextrose. In: Rowe R C, Sheskey P J and Owen S C, eds. Handbook of Pharmaceutical Excipients. 5th ed. Washington D.C.: American Pharmaceutical Association; 2005: 231-233; Price J C. Glycerin. In: Rowe R C, Sheskey P J and Owen S C, eds. Handbook of Pharmaceutical Excipients. 5th ed. Washington D.C.: American Pharmaceutical Association; 2005: 301-303; Price J C. Glycerin. In: Rowe R C, Sheskey P J and Owen S C, eds. Handbook of Pharmaceutical Excipients. 5th ed. Washington D.C.: American Pharmaceutical Association; 2005: 301-303; Armstrong N A. Mannitol. In: Rowe R C, Sheskey P J and Owen S C, eds. Handbook of Pharmaceutical Excipients. 5th ed. Washington D.C.: American Pharmaceutical Association; 2005: 449-453; Owen S C. Sodium Chloride. In: Rowe R C, Sheskey P J and Owen S C, eds. Handbook of Pharmaceutical Excipients. 5th ed. Washington D.C.: American Pharmaceutical Association; 2005: 671-674. Mannitol is an example of a GRAS listed ingredient accepted for use as a food additive in Europe, included in the FDA Inactive Ingredients Database (IP, IM, IV, and SC injections; infusions; buccal, oral and sublingual tablets, powders and capsules; ophthalmic preparations; topical solutions), included in nonparenteral and parenteral medicines licensed in the UK and included in the Canadian Natural Health Products Ingredients Database. A 5.07% w/v aqueous solution is isoosmotic with serum.

Hypotonic formulations, typically in the range of 0-220 mOsm/kg, provide rapid delivery of nanoparticle vehicle to uniformly distribute and penetrate to the bladder tissue, rather than in the bladder lumen. Blood plasma is generally considered isotonic at ˜300 mOsm/kg; a higher osmolality in the colon; and a higher osmolality about 400-800 mOsm/kgin urine.

4. Formulations

For those embodiments where the one or more therapeutic, prophylactic, and/or diagnostic agents are encapsulated within a polymeric nanoparticle and/or associated with the surface of the nanoparticle, the percent drug loading is from about 1% to about 80%, from about 1% to about 50%, preferably from about 1% to about 40% by weight, more preferably from about 1% to about 20% by weight, most preferably from about 1% to about 10% by weight. The ranges above are inclusive of all values from 1% to 80%. For those embodiments where the agent is associated with the surface of the particle, the percent loading may be higher since the amount of drug is not limited by the methods of encapsulation. In some embodiments, the agent to be delivered may be encapsulated within a nanoparticle and associated with the surface of the particle.

### Liquid Formulations

Liquid formulations contain one or more assembled particles suspended in a liquid pharmaceutical carrier.

Suitable liquid carriers include, but are not limited to, distilled water, de-ionized water, pure or ultrapure water, saline, and other physiologically acceptable aqueous solutions containing salts and/or buffers, such as phosphate buffered saline (PBS), Ringer's solution, and isotonic sodium chloride, or any other aqueous solution acceptable for administration to an animal or human.

Preferably, liquid formulations are hypotonic relative to ladder physiological fluids and of approximately the same pH, ranging from about pH 4.0 to about pH 7.4, more preferably from about pH 6.0 to pH 7.0. The liquid pharmaceutical carrier can include one or more physiologically compatible buffers, such as a phosphate buffers. One skilled in the art can readily determine a suitable saline content and pH for an aqueous solution for pulmonary administration.

Liquid formulations may include one or more suspending agents, such as cellulose derivatives, sodium alginate, polyvinylpyrrolidone, gum tragacanth, or lecithin. Liquid formulations may also include one or more preservatives, such as ethyl or n-propyl p-hydroxybenzoate.

Formulations may be prepared using one or more pharmaceutically acceptable excipients, including diluents, preservatives, binders, lubricants, disintegrators, swelling agents, fillers, stabilizers, and combinations thereof. Liquid formulations may also contain minor amounts of polymers, surfactants, or other excipients well known to those of the art. In this context, “minor amounts” means no excipients are present that might adversely affect the delivery of assembled gel compositions to targeted tissues, e.g. through circulation.

### Dry Powder Formulations and Kit

In some forms, the gelators, stabilizing agents, and optionally one or more therapeutic, prophylactic, and diagnostic agents are formulated in dry powder forms as finely divided solid formulations. The dry powder components can be stored in separate containers, or mixed at specific ratios and stored. In some embodiments, suitable aqueous and organic solvents are included in additional containers. In some embodiments, dry powder components, one or more solvents, and instructions on procedures to mix and prepare assembled nanostructures are included in a kit. Alternatively, stabilized, assembled particles, nanoparticles or bulk gel thereof are dried via vacuum-drying or freeze-drying, and suitable pharmaceutical liquid carrier can be added to rehydrate and suspend the assembled nanostructures or gel compositions upon use.

Dry powder formulations are typically prepared by blending one or more gelators, stabilizing agents, or active agents with one or more pharmaceutically acceptable carriers. Pharmaceutical carrier may include one or more dispersing agents. The pharmaceutical carrier may also include one or more pH adjusters or buffers. Suitable buffers include organic salts prepared from organic acids and bases, such as sodium citrate or sodium ascorbate. The pharmaceutical carrier may also include one or more salts, such as sodium chloride or potassium chloride.

The dry powder formulations can be suspended in the liquid formulations to form assembled particles or nanoparticles thereof, and administered systemically or regionally using methods known in the art for the delivery of liquid formulations.

### Injectable Formulations

In some embodiments, the stabilized, assembled particles are formulated for parenteral delivery, such as injection or infusion, in the form of a solution or suspension. The formulation is preferably administered directly to the bladder or tissue to be treated. Formulations can be prepared as aqueous compositions using techniques is known in the art. Typically, such compositions can be prepared as injectable or unfusiable formulations, for example, solutions or suspensions; solid forms suitable for using to prepare solutions or suspensions upon the addition of a reconstitution medium prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized gelators, stabilizing agents, and/or active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzyl peroxide, benzethonium chloride, benzyl alcohol, cetylpyridinium chloride, chlorobutanol, phenol, phenylethyl alcohol, and thimerosal.

Formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and process for preparing tablets and capsules and delayed release dosage forms of tablets, capsules, and granules.

### III. Method of Making

The particles are generally formed via the assembly or association (non-covalently or covalently) between one or more polymer and agent, such that the particles are capable of dissolution or disassembly to release the agent in the bladder tissue, leading to an improved absorption and retention of agents in bladder. These particles may be nanosized or have a size in the micrometer range. They are generally in the form of micelles, colloids, liposomes, vesicles, nanodroplets nano-structured hydrogel, nanocrystals, or nanosuspension.

In some embodiments of delivering metal-based agents, polymer is incubated with the agent to allow formation of polymer→metal (e.g., O→Pt) coordination, generally via ligand substitution reaction.

In some embodiments, nanosuspension or nanocrystal is formed by mixing therapeutic agents with a polymer or matrix material; and following either a bottom-up approach to assembling precipitation, microemulsion, or melt emulsification, or a top-down approach to disintegrating larger particles into nanoparticles via, e.g., high-pressure homogenization and milling. Preparation of nanosuspension is simple and applicable to water insoluble drugs. A nanosuspension provides improved solubility and bioavailability, as well as alters the pharmacokinetics of drug and thus improves drug safety and efficacy.

Other techniques for making particles include solvent evaporation, solvent removal, spray drying, phase inversion, low temperature casting, and nanoprecipitation. Suitable methods of particle formulation are briefly described below in the Examples. Pharmaceutically acceptable excipients, including pH modifying agents, disintegrants, preservatives, and antioxidants, can optionally be incorporated into the particles during particle formation.

### IV. Methods of Using

Treatment options for bladder cancer depend on a number of factors, including the type of cancer, grade of the cancer and stage of the cancer, which are taken into consideration along with your overall health and your treatment preferences.

Bladder cancer treatment may include:

Surgery, to remove cancerous tissue

Chemotherapy in the bladder (intravesical chemotherapy), to treat tumors that are confined to the lining of the bladder but have a high risk of recurrence or progression to a higher stage

Reconstruction, to create a new way for urine to exit the body after bladder removal

Chemotherapy for the whole body (systemic chemotherapy), to increase the chance for a cure in a person having surgery to remove the bladder, or as a primary treatment in cases where surgery isn't an option

Radiation therapy, to destroy cancer cells, often as a primary treatment in cases where surgery isn't an option or isn't desired

Immunotherapy, to trigger the body's immune system to fight cancer cells, either in the bladder or throughout the body

Approaches to bladder cancer surgery might include:

Transurethral resection of bladder tumor (TURBT). TURBT is a procedure to remove bladder cancers confined to the inner layers of the bladder, those which aren't yet muscle-invasive cancers. During the procedure, a surgeon passes a small wire loop through a cystoscope and into the bladder. The wire loop burns away cancer cells using an electric current.

Alternatively, a high-energy laser may be used to destroy the cancer cells. As part of the TURBT procedure, doctors may recommend a one-time injection of chemotherapy into the bladder to destroy any remaining cancer cells and to prevent a tumor from coming back. The chermotherapeutic agent remains in the bladder for a short period of time, up to an hour, and then is drained.

Cystectomy is surgery to remove all or part of the bladder. Partial cystectomy may only be an option if cancer is limited to one area of the bladder that can easily be removed without harming bladder function. A radical cystectomy removes the entire bladder, part of the ureters and surrounding lymph nodes. In men, radical cystectomy typically includes removal of the prostate and seminal vesicles. In women, radical cystectomy also involves removal of the uterus, ovaries and part of the vagina. Cystectomy carries a risk of infection and bleeding. In men, removal of the prostate and seminal vesicles may cause erectile dysfunction. In women, removal of the ovaries causes infertility and premature menopause.

After a radical cystectomy, the surgeon must create a new way for urine to leave your body (urinary diversion). One option for urinary diversion is neobladder reconstruction. Your surgeon creates a sphere-shaped reservoir out of a piece of your intestine. This reservoir, often called a neobladder, sits inside your body and is attached to your urethra. In most cases, the neobladder allows one to urinate normally. A small number of people with a neobladder have difficulty emptying the neobladder and may need to use a catheter periodically to drain all the urine from the neobladder. For an ileal conduit, the surgeon creates a tube (ileal conduit) using a piece of the intestine. The tube runs from your ureters, which drain your kidneys, to the outside of your body, where urine empties into a urostomy bag.

Chemotherapy treatment for bladder cancer usually involves two or more chemotherapy drugs used in combination.

Chemotherapy drugs can be given:

Through a vein (intravenously)

Via a tube passed through the urethra directly to your bladder (intravesical therapy)

Chemotherapy is frequently used before bladder removal surgery to increase the chances of curing the cancer. Chemotherapy may also be used to kill cancer cells that might remain after surgery. Chemotherapy is sometimes combined with radiation therapy in very select cases as an alternative to surgery.

Intravesical chemotherapy may be the primary treatment for superficial bladder cancer, where the cancer cells affect only the lining of the bladder and not the deeper muscle tissue. Immunotherapy may be administered as intravesical therapy for superficial bladder cancer.

Radiation therapy uses high-energy beams aimed at the cancer to destroy the cancer cells. Radiation therapy is sometimes combined with chemotherapy as an alternative to surgery

Immunotherapy works by signaling the body's immune system to help fight cancer cells. Immunotherapy for bladder cancer often is administered through the urethra and directly into the bladder (intravesical therapy). One such immunotherapy drug used to treat bladder cancer is Bacillus Calmette-Guerin (BCG), which is a vaccine used to protect against tuberculosis. Another immunotherapy drug is a synthetic version of interferon called interferon alfa-2b (Intron A), sometimes used in combination with BCG.

Atezolizumab (Tecentriq) is an immunotherapy option for locally advanced or metastatic bladder cancer that does not respond to or becomes worse after chemotherapy. An intravenous (IV) medication, the drug works by triggering the body's immune system to attack the cancerous tumor.

The formulations containing the particles described herein can be used with any of these drugs, chemotherapeutics, radioactive pellets, and/or immunotherapy for administration to the bladder or other urothelium-containing organs such as renal pelvis, urinary bladder, the ureters, the superior urethra, or the prostatic and ejaculatory ducts of the prostate, in an effective amount to alleviate or prevent or diagnose one or more symptoms, wherein the formulation is hypotonic to enhance uptake and penetration of the particles through the bladder or intended tissue.

Exemplary diseases or disorders to be treated with the formulation include, but are not limited to, infections, inflammation, cancer, bladder obstruction, cystitis, diverticulum of the bladder, overflow incontinence, stress incontinence, urge incontinence, and vesicoureteral reflux.

The formulation can also be administered through various known regional delivery techniques, including injection, implantation, instillation, and topical application to the bladder or other urothelium-containing organs. In other embodiments, it may also be administered systemically.

The present invention will be further understood by reference to the following non-limiting examples.

### Examples

### Example 1: Hypotonic Medium Facilitated Nanoparticle Uptake by Bladder; and “Soft” (“Dissolvable”) Nanoparticles Achieved Non-Reversible Drug Penetration into Bladder Tissue Before Reversal of Osmotic Imbalance

The bladder epithelium is exposed to urine with a composition widely different from that of plasma (isotonic), and must maintain osmotic equilibrium using a mechanism that does not involve water transfer across the epithelial surface. Unlike other mucosal surfaces such as the colon or the vagina, which absorb water across the epithelium to re-establish osmotic equilibrium, the bladder epithelium forms fluid-filled vesicles that can reversibly fuse with the bladder epithelium.

Materials and Methods

Using non-degradable polystyrene nanoparticles, the impact of medium tonicity on delivery of nanoparticles (NPs) to the bladder was assayed.

Results

Microscopic images demonstrated the uptake by the bladder of nanoparticles in a hypotonic fluid, whereas the majority of nanoparticles administered in saline remained in the lumen of the bladder of mice.

This NP uptake process was reversible: when the bladder refilled with urine and the animals urinated as normal, the nanoparticles were quickly voided (within 30 min). This phenomenon was also previously demonstrated by others (Chang et al.) for small molecule fluorophores.

Thus, the use of a “soft” nanoparticle, e.g., one formed from ionic interactions or a drug nanocrystal, which could fall apart and deliver drug into the tissue before the reversal of the osmotic imbalance, was tested in combination with a hypotonic vehicle and believed to be beneficial for drug delivery into bladder tissue. It was observed that a model drug fluorescein nanocrystal, formulated as a dissolvable nanocrystal, penetrated deep into the bladder tissue, which was non-reversible, and was uniformly distributed from a hypotonic vehicle.

### Example 2: Cisplatin “Soft” Nanoparticles (Formed Via Ionic Interactions) with and without Low and High Grafting Density of PEG are Effective in Killing Cancer Cells In Vitro

Materials & Methods

Poly-L-aspartic acid (PAA) with molecular weight (MW) of 27 kDa and linear PEG-PAA (MW: 5 kDa/27 kDa) were purchased from Alamanda Polymers (Huntsville, Ala.). According to the vendor, poly-L-aspartic acid sodium salt is a negatively charged synthetic polyamino acid having one Na per aspartic acid unit. Cis-diamminedichloroplatinum (cisplatin or CDDP, 99.9%), silver nitrate, and 100 kDa Amicon-Ultra-2 mL filters were purchased from Sigma-Aldrich (St. Louis, Mo.). 20 kDa and 50 kDa dialysis cassettes were obtained from Spectrum Labs (Rancho Dominguez, Calif.). AlexaFluor 647-cadaverine was purchased from Thermo Fisher Scientific (Waltham, Mass.). PEG (NH2-PEG-OCH3; MW: 5 kDa) was purchased from Creative PEGworks (Winston Salem, N.C.) and 1-ethyl-3-(3-dimethylaminopropyl) carbodiimide (EDC) was purchased from Invitrogen (Carlsbad, Calif.).

Self-Assembly Formation of PAA-CDDP Nanoparticles (NPs)

PAA and CDDP were dissolved in nuclease-free water at a concentration of 1 mg/ml and 1.25 mg/ml, respectively. PAA solution was mixed with CDDP solution in 1:1 volume ratio, and the mixture was continuously stirred and protected from light. After 3 d at room temperature, self-assembled PAA-CDDP NPs were formed. PAA-CDDP NPs were washed once with ultrapure water using 100 kDa Amicon-Ultra centrifugal filters to remove excess CDDP and PAA.

For formulation of fluorescent PAA-CDDP-NPs (AF647-PAA-CDDP NPs), Alexa Fluor® 647 dye-PAA conjugate and PAA were dissolved in water at a ratio of 2:3 to obtain a total concentration of 1 mg/ml. The remaining process for NP formation was the same as described for PAA-CDDP NPs.

Formation of PEG-PAA-CDDP NPs

PEG_{low}-PAA-CDDP NPs were composed of commercially available linear PEG-PAA (PEG:PAA 1:1). PEG_{high}-PAA (PEG:PAA 10:1) was synthesized as described in Paraskar A S, et al., Proc Natl Acad Sci USA, 107(28):12435-12440, and Sengupta P, et al., Proc Natl Acad Sci, 109(28):11294-11299.

### Nanoparticle Characterization

Following purification, all nanoparticle formulations were characterized for particle size, polydispersity index, and ζ-potential using a Malvern Zetasizer Nano Z590. Particle size measurements were carried out at a scattering angle of 173°. For ζ-potential, nanoparticles were diluted with 10 mM NaCl solution at pH 7. Transmission electron microscopy (TEM) experiments were performed on a Hitachi H7600 microscope. CDDP content (drug loading) was determined by atomic absorption spectroscopy (AAS). Briefly, 10 μl of nanoparticle stock solution was diluted with 990 μl of 2.2% nitric acid, and platinum (Pt) content was measured using AANALYST™ 800 atomic absorption spectrometer (Perkin-Elmer Inc., Waltham, Mass.). A 1 ml volume of nanoparticle stock solution was then lyophilized in pre-weighed tubes and subsequently weighed to determine the mass of lyophilized nanoparticles. Drug loading was calculated and reported as wt % (wt cisplatin/wt particles).

### In Vitro Cytotoxicity Assay

In vitro cytotoxicity of CDDP, oxaliplatin, PAA-CDDP NPs, PEG_{low}-PAA-CDDP NPs and PEG_{high}-PAA-CDDP NPs was evaluated using a superficial bladder cancer cell line (RT4) in triplicate. RT4 cells were purchased from the American Type Culture Collection (ATCC, Manassas, Va.) and cultured in McCoy's 5A (Thermo Fisher Scientific, Waltham, Mass.) at 37° C. under 5% CO₂. Cells were seeded in 96-well tissue-treated culture plates at a density of 5×10³ cells in 100 μl of complete medium and cultured overnight. Then, the supernatant was removed, and a known concentration of CDDP, oxaliplatin, or CDDP NPs (100 μl) was added into the wells. After 72 hr, cell viability was measured using CellTiter-Glo Luminescent Cell Viability Assay Kit (Promega, Madison, Wis.) according to manufacturer's protocol. Acquired data was analyzed to depict sigmoidal dose-response curve with Graph Pad Prism 5 (Graph Pad Software Inc., San Diego, Calif.) using the equation log(inhibitor) vs. response-variable slope.

Results

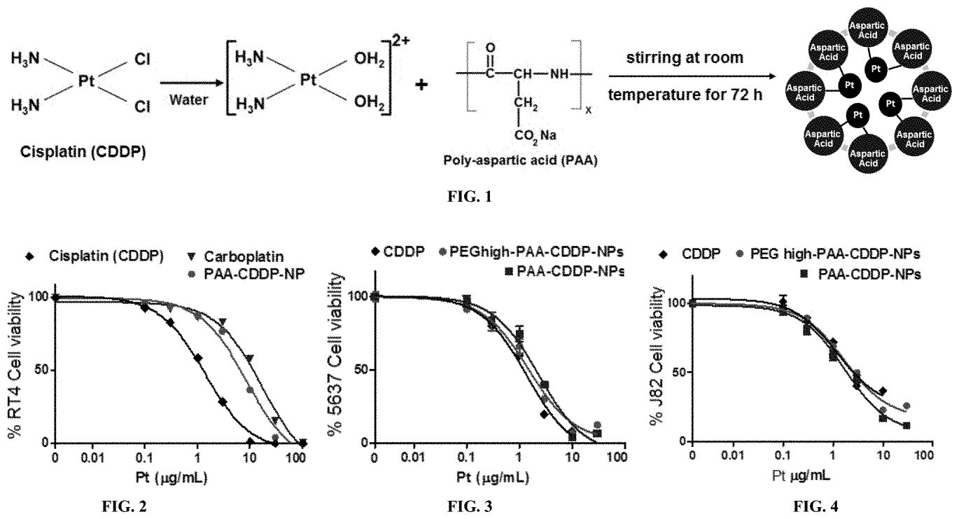

FIG. 1 shows a reaction scheme of assembly of PAA with CDDP. The complexed structure between CDDP and monomers of PAA is shown below:

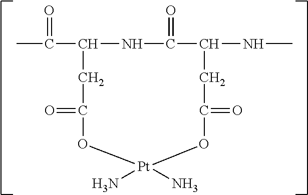

PAA-CDDP NPs were 140±5 nm in size with a narrow size distribution (polydispersity index 0.2±0.1) and negative surface charge (ζ-potential: −35±2.0 mV) due to the presence of unreacted carboxylate groups on the polymer backbone. PEG_{low}-PAA-CDDP NPs and PEG_{high}-PAA-CDDP NPs were smaller in size (<50 nm) and exhibited a near-neutral surface charge (Table 1).

TABLE 1
Particle size for PEG-PAA-CDDP NPs and fluorescent NPs.

| | | | % CDDP loading |
|---|---|---|---|
| | | | (wt cisplatin/ |
| Formulation | Size (PDI) | Zeta potential | wt particles) |
| CDDP-PAA-NPs | 140 ± 4 nm | −38.3 ± 2 mV | 17.5 ± 1.6% |
| | (0.23 ± 0.01) | | |
| PEG_{low}- | 49 ± 2 nm | −3.9 ± 0.1 mV | 34.6 ± 2% |
| PAA-CDDP-NPs | (0.11 ± 0.02) | | |
| PEG_{high}- | 45 ± 2 nm | −3.27 ± 0.48 mV | 40 ± 5% |
| PAA-CDDP-NPs | (0.15 ± 0.02) | | |
| AF647-PAA- | 110 ± 1 nm | −26.7 ± 2 mV | Not determined |
| CDDP-NPs | (0.27 ± 0.03) | | |

The partitioning of PEG to the nanoparticle surface likely controlled the rate and extent of reaction between CDDP and the carboxylate groups of PAA, yielding smaller nanoparticles shielding the nanoparticle surface. These observations are in accordance with published data in Nishiyama N, et al., J. Controlled Release, 74(1-3):83-94 (2001).

TEM image confirmed all types of CDDP NPs were spherical in shape and showed high loading of CDDP (Table 1).

### CDDP NPs Retained Anti-Cancer Activity of CDDP

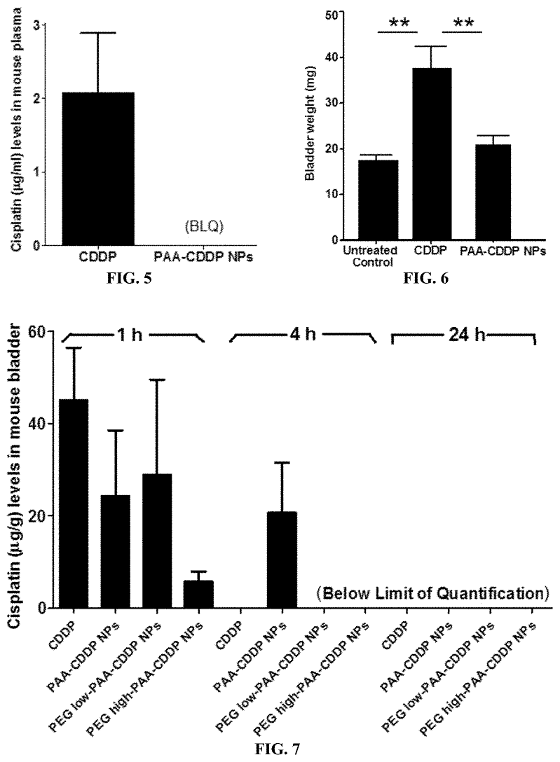

Drugs like carboplatin are prepared by conjugation of platinum with carboxylate groups (of carbolic acid), and, due to this conjugation process, show considerably lower cytotoxicity to cancer cells in vitro compared to CDDP (Alberts D, et al., Oncologist, 3(1):15-34 (1998); Powles T, et al., Urol Int, 79(1):67-72 (2007)). As CDDP NPs were formed via conjugation of CDDP with the carboxylate groups of PAA, the potential loss in potency of the anti-cancer activity of CDDP was evaluated. The cytotoxicity of CDDP, carboplatin, and various CDDP NPs in vitro against a superficial bladder cancer cell line (RT4) was assayed. All types of CDDP NPs showed IC₅₀ values that were increased compared to CDDP, but lower than carboplatin (FIG. 2, Table 2), indicating that more of the anti-cancer activity of free CDDP was retained in the NP formulations compared to carboplatin. PAA-CDDP NPs and PEG_{high}-PAA-CDDP NPs also showed similar cytotoxicity to CDDP in killing high grade invasive bladder cancer cell lines such as 5367 and J82 (FIGS. 3 and 4).

TABLE 2
IC₅₀ values of various formulations.

| | RT4 cells-IC₅₀ |
|---|---|
| Formulation | (μg/ml of Pt) |
| Cisplatin | 1.39 ± 0.03 |
| Carboplatin | 17.18 ± 0.056 |
| PAA-CDDP-NPs | 8.57 ± 0.051 |
| PEG_{low}- | 6.48 ± 0.049 |
| PAA-CDDP-NPs | |
| PEG_{high}- | 9.03 ± 0.064 |
| PAA-CDDP-NPs | |

### Discussion

Nanoparticles composed of linear polyaspartic acid (PAA) polymer complexed with cisplatin (CDDP) formed spontaneously in water. The physical interaction between the PAA and cisplatin is believed to be reversible and would result in dissociation of the drug for release. This feature was believed to be beneficial for drug release and penetration into the bladder tissue before the reversal of osmotic imbalance, thereby before the voiding (e.g., within 30 minutes) of nanoparticle vehicles when the bladder refills with urine and the animals urinate as normal. A “hard sphere” nanoparticle is generally reversibly exocytosed by the bladder when the bladder is filled with urine.

### Example 3: CDDP NPs Reduced Systemic Exposure and Local Toxicity of CDDP In Vivo (Mouse and Rat)

Materials & Methods

### Assessment of Toxicity in Mice

Female CF-1 mice (age 8 weeks) were purchased from Harlan (Indianapolis, Ind.) and acclimated in the animal facility for 4 weeks. Mice were randomly divided into different groups (n≥5). Mice were anesthetized with an isoflurane vaporizer and nose cone system and catheterized using polyethylene tubing mounted on a 30G needle. After catheterization, the bladder was emptied of urine by aspiration and/or gentle pressure on the abdomen. Then, 100 μl of CDDP solution, PAA-CDDP NPs, PEG_{low}-PAA-CDDP NPs or PEG_{high}-PAA-CDDP NPs at 0.7 mg/ml CDDP content was instilled into the bladder by intravesical administration. Mice were maintained under anesthesia for 1 h and then allowed to wake up. To assess systemic exposure, mice were euthanized at 1, 4, and 24 h and plasma was obtained for analysis of CDDP content. To assess local toxicity of CDDP solution and CDDP NPs, mice received a total of three intravesical doses spaced one week apart. Bladder tissues were obtained 24 h after the third dose, photographed, and weighed.

Bladder Uptake and Retention of Fluorescent NPs in Mice

AF647-PAA-CDDP NPs (CDDP content: 0.7 mg/ml) were instilled into the bladders by intravesical administration of mice (n=3 per group) as described above. Mice were maintained under anesthesia for 1 h and then allowed to wake up. Bladder tissues were obtained at 1 h and 4 h (3 h ambulatory time) to evaluate uptake and retention of AF647-PAA-CDDP NPs (thus representing PAA-CDDP NPs). Bladder tissues were dipped in phosphate buffered saline, gently squeezed with forceps to remove any residual fluid, transferred to plastic cryomolds filled with Optimal Cutting Temperature (OCT) medium (Tissue-Tek), and subsequently frozen in liquid nitrogen. Frozen bladders were cryosectioned into 6 μm sections with a cryostat (Leica CM 3050S, Leica) and mounted onto positively charged microscope slides. Slides were washed with 1× tris-buffered saline (TBS, Mediatech), dried, stained with DAPI (ProLong® Gold antifade reagent with DAPI, Invitrogen), sealed with a cover slip, and imaged using a Zeiss confocal 710 laser scanning microscope in the DAPI and Cy5 channels.

### Bladder Uptake and Retention in Rats

Female Fischer 344 rats (age 7 weeks) were purchased from Harlan (Indianapolis, Ind.) and acclimated in the animal facility for 1 week. Before starting the experiment, rats were randomly divided into different groups (n≥5). Rats were anesthetized with an isoflurane vaporizer and nose cone system and catheterized using a 20G angiocatheter sheath. After catheterization, the bladder was voided using aspiration and/or gentle pressure on the abdomen. Then, 300 μl of CDDP solution, PAA-CDDP NPs, PEG_{low}-PAA-CDDP NPs, or PEG_{high}-PAA-CDDP NPs (CDDP content 0.7 mg/ml) was instilled by intravesical administration. Rats were maintained under anesthesia for 1 h and then allowed to wake up. Plasma and bladder tissues were obtained at 1 and 4 h for analysis of CDDP content. Bladder tissues were dipped in PBS and gently squeezed with forceps to remove any residual fluid.

### Analysis of Released CDDP Content in Plasma and Bladder Tissue Following Intravesically Administration in Rats

200 μl of plasma or bladder were transferred to a clean borosilicate glass cell culture tube, and 1 ml of 22% nitric acid was added. All samples were heated to 90° C. to digest plasma proteins or bladder tissues and to extract platinum. The solution was heated until a dry yellow film was obtained. Then, 1 ml of 2.2% nitric acid added to dissolve the dried residue containing platinum. CDDP content in the solution was analyzed by AAS. The lowest limit of quantification for CDDP was 250 ng/ml (<1% of the administered dose).

### In Vivo Efficacy Assessment in N-Methyl-N-Nitrosourea (MNU) Induced Rat Bladder Carcinogenesis Model

The N-methyl-N-nitrosourea (MNU) rat model of bladder cancer was chosen because it recapitulates human non-muscle invasive bladder cancer, with carcinoma in situ (CIS), non-invasive papillary carcinoma (Ta), and high grade invasive papillary carcinoma (T1) histologies, and progresses from dysplasia to NMIBC to MIBC (Steinberg G D, et al., Cancer Res, 50(20):6668-74 (1990)). This animal model progresses from dysplasia to NMIBC between about week 8 and about week 16, making it a desirable time to initiate treatment and a good recapitulation of a chemopreventative treatment model. Additionally, the MNU model represents an outbred strain of NMIBC, as opposed to an inbred strain utilizing immortal clones of genetically identical tumor strains. While an inbred strain derived from a cell line provides consistent tumor size and growth, its homogeneity indicates that any treatment effects translate to only a small group of genetically similar tumor subtypes. Since no reliable xenograft model was believed to be present of NMIBC that progresses to MIBC in a temporal time-dependent fashion similar to human urothelial cancer, a carcinogen model was chosen to recapitulate the heterogeneity of human disease, study the antineoplastic and immunologic effects of treatment, and reflect the typical progression of non-muscle to muscle invasive cancer. In this model, the presence of NMIBC progresses from about week 8 to about week 15, at which point all rats have NMIBC.

Fischer 344 female rats (age 7 weeks) were anesthetized with an isoflurane vaporizer and nose cone system. After complete anesthesia and preparation of the surgical area, a 20G angiocatheter (BD) was placed into the rat's urethra and the bladder was emptied of urine. MNU (1.5 mg/kg MNU dissolved in 0.30 ml of 0.9% saline, pH 6.0) was then instilled into the bladder under 45 min of continued sedation to prevent spontaneous micturition and allow absorption. In order to explore the potential for treatment to prevent recurrence, treatment was initiated from weeks 8-14. Treatment groups (n=5 per group) included CDDP and PAA-CDDP NPs dosed weekly. All formulations were delivered at a concentration of 0.7 mg/ml in 300 μl. Rats were sacrificed at week 15 for histopathologic analysis of bladder tissue. Bladders were formalin fixed, paraffin embedded, sectioned, and stained with hematoxylin-eosin for classification according to the World Health Organization/International Society of Urological Pathology consensus. Tumor staging was performed in a blinded fashion by a board certified genitourinary pathologist.

### Immunohistochemical Staining

For immunohistochemical staining, high-temperature antigen retrieval (18-23 psi/126° C.) was performed by immersing the slides in Trilogy (Cell Marque, Hot Springs, Ark.). Endogenous peroxidase activity was blocked for 5 min in using Dual Endogenous Enzyme Block (Dako S2003). Primary Antibody used included KI 67 (Abcam;ab16667). Slides were stained with Impact DAB (Vector Labs, Burlingame, Calif., USA) for 3 min and counterstained with haematoxylin (Richard-Allen, Kalamazoo, Mich., USA). For each section, Ki67+, cells were counted in 10 random 400× fields.

### Statistical Analysis

Statistical analysis was performed using Prism 5 (GraphPad). One-way ANOVA tests with Bonferroni adjustment for multiple comparisons were conducted and results were considered statistically significant at P≤0.05.

Results

### 1. Intravesically Administered CDDP NPs Reduced Systemic Exposure to CDDP and Local Toxicity of CDDP in Mice.

CDDP has been shown to cause systemic and local toxicity in humans. Thus, the systemic CDDP exposure and local toxicity of CDDP and CDDP NPs after intravesical administration were evaluated. Intravesical administration of CDDP solution in mice resulted in rapid systemic exposure at 1 h (˜1.5 μg/ml in plasma), whereas none of the mice dosed with CDDP NPs had detectable amounts of CDDP in their plasma (FIG. 5). At 4 h and 24 h, CDDP was below the limit of quantification (BLQ, <250 ng/ml) in plasma for all treatment groups. Three weekly intravesical administrations of the CDDP solution led to a significant increase in the bladder weight due to hyperplasia compared to PAA-CDDP NPs and sham controls (FIG. 6). In contrast, the bladders of mice that received three weekly doses of PAA-CDDP NPs were of similar weights to the bladders of sham treated control mice (FIG. 6). The increases in bladder tissue weights after CDDP treatment were also confirmed by gross observations of bladder size. This indicates significant tissue hyperplasia and toxicity with intravesically administered CDDP in solution, which could explain the anaphylactic reactions associated with intravesical CDDP in human clinical trials and may be the reason CDDP is not typically used for intravesical therapy.

### 2. Intravesically Administered PAA-CDDP NPs Retained in Mouse Bladder for at Least 4 Hours.

When the mice were maintained under anesthesia and no voiding of the bladder was observed, CDDP levels in the mouse bladders were similar at 1 h post intravesical administration among CDDP, PAA-CDDP NPs, and PEG_{low}-PAA-CDDP NPs (FIG. 7). In contrast, CDDP levels were much lower in bladder tissue at 1 h after intravesical administration of PEG_{high}-PAA-CDDP NPs (FIG. 7). At 4 h after intravesical administration (3 h of ambulatory time), only PAA-CDDP NPs treated mice had detectable levels of CDDP in their bladders. Moreover, the concentration of CDDP 4 h after PAA-CDDP NPs administration was >50% of the amount observed at 1 h (FIG. 7), indicating that the PAA-CDDP NPs were well-retained in the bladder after urination. The retention of PAA-CDDP NPs in the bladder after urination was confirmed by fluorescent imaging of fluorescently-labeled CDDP-PAA NPs with similar physicochemical properties to CDDP-PAA NPs (Table 1).

Confocal imaging of bladder sections 1 h after intravesical administration of fluorescent PAA-CDDP NPs showed uniform distribution and subepithelial accumulation at the 1-hour time point as well as the 4-hour time point, indicating the retention of PAA-CDDP NPs in bladder for at least 4 hours. At 24 h, CDDP levels in the bladder were below limit of quantification (BLQ) for all formulations (FIG. 7).

Therefore, the results confirmed the hypothesis that CDDP NPs may increase local delivery and retention of active agent while decreasing systemic CDDP exposure. It was likely that the sustained release of CDDP from NPs led to the decreased systemic exposure and decreased local toxicity compared to intravesical administration of CDDP solution. It was also likely that poly-amino acid backbone, the surface charge, and the size played a role in the processes of the uptake and retention of PAA-CDDP NPs in the bladder.

3. PAA-CDDP NPs Improved Uptake and Retention of CDDP in Rats than Free CDDP in Solution.

Prior to initiating studies for in vivo efficacy in a rat model of NMIBC, PAA-CDDP NPs were examined for the bladder uptake and retention compared to a CDDP solution. FIG. 8 shows PAA-CDDP NPs provided significantly increased CDDP concentration (6-fold increase) in rat bladders at 1 h after intravesical administration compared to CDDP solution. Similarly, CDDP levels were below the level of detection in rat bladders 4 h after administration of CDDP solution, whereas the CDDP concentration 4 h after PAA-CDDP NPs dosing was maintained at 45% of the concentration at 1 h.

Similar to what was observed in mice (FIG. 7), FIG. 8 shows CDDP levels in rat bladder following intravesical administration at 1 h were lower for PEG_{low}-PAA-CDDP NPs and PEG_{high}-PAA-CDDP NPs, compared to PAA-CDDP NPs without PEG; and the CDDP levels were below limit of quantification/detection in bladder tissues 4 h after dosing for either high or low density PEGylated NP formulation. An explanation for this phenomenon was the additional stability provided by PEG led to decreased drug delivery to the bladder tissue prior to the re-establishment of osmotic equilibrium. This was also in contrast to previous nanoparticle studies for delivery to other mucosal surfaces where a dense PEG coating was needed for crossing the mucus barrier. Here, the bladder does not have a loose, luminal mucus coating that requires particles to effectively penetrate.

From both studies in mice and rats, the delivery of CDDP via PAA-CDDP NPs was shown to be not reversible. Formulations were infused by intravesical administration for 1 hour, then both mice and rats were ambulatory and mobile for an additional 3 hours (to allow urination and bladder voiding). The levels of CDDP at the 4-hour time point in both mice and rats (4 h in FIGS. 7 and 8, respectively) via CDDP-PAA NPs were much greater, compared to those administered with free CDDP solution, (the latter below the limit of quantitation, BLQ).

### 4. In Vivo (Rat) Efficacy in Preventing Progression of Bladder Tumor.

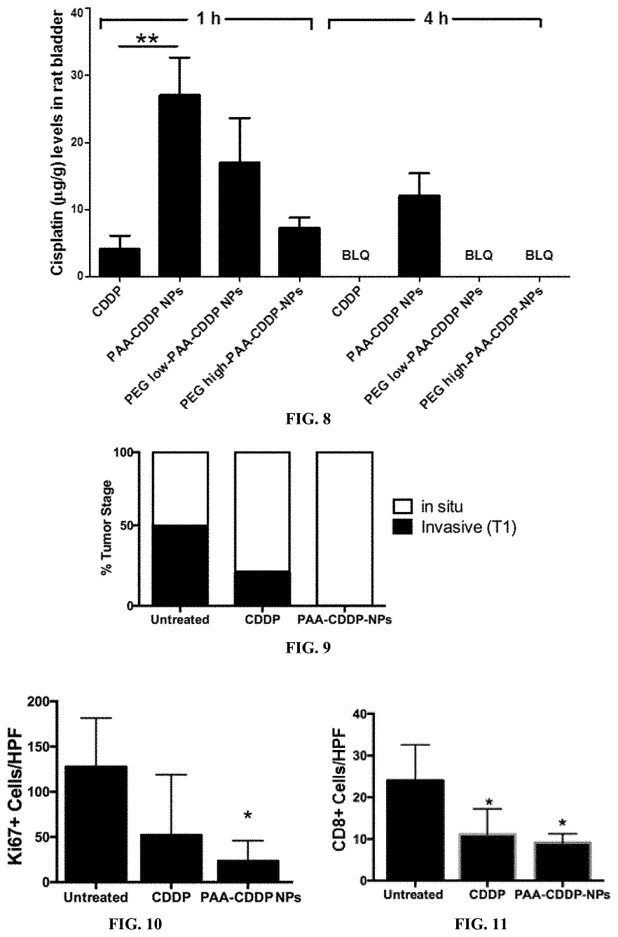

Histopathological and immunohistochemical analysis of rat bladder tissues showed significant progression of bladder cancer in untreated controls, with evidence of carcinoma in situ (CIS) or high grade invasive papillary carcinoma (T1) in all specimens, validating the MNU-induced rat bladder carcinogenesis model. Gross histopathological analysis of bladder tissues showed rats treated with intravesical PAA-CDDP NPs had no signs of T1 grade tumors, whereas just 20% of rats treated with CDDP had T1 high grade tumors in the bladder (FIG. 9). This data demonstrated intravesical PAA-CDDP NPs prevented progression of bladder tumors to the invasive T1 grade.

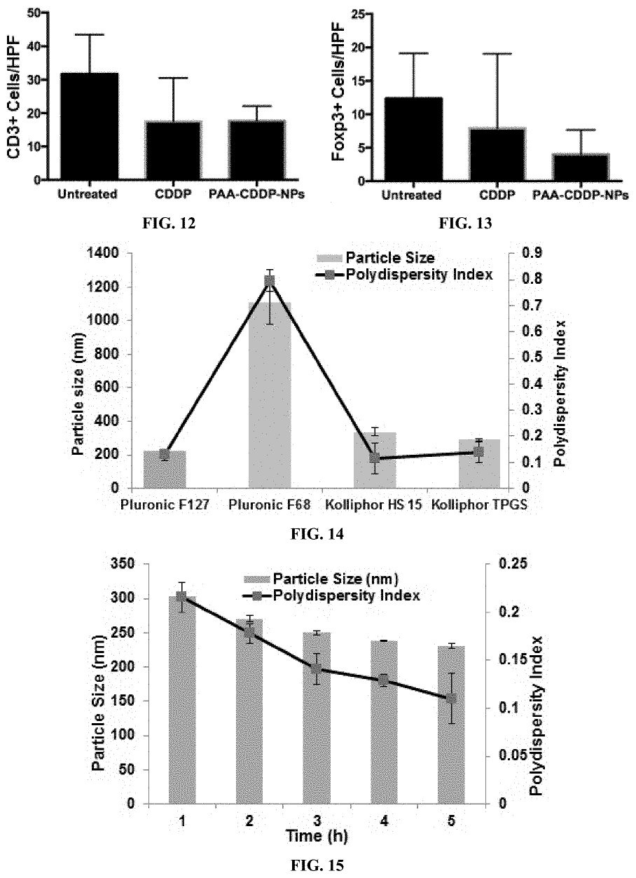

The tumor grading was supported by immunohistochemical staining and analysis of bladder sections. Bladder tissues from rats treated with intravesical PAA-CDDP NPs had significantly decreased Ki67+ staining, a marker of cancer cell proliferation, compared to untreated controls (P<0.05). Bladder tissues from rats treated with intravesical CDDP solution had decreased Ki67+ staining, though statistically indistinguishable from the untreated control group (FIG. 10). In addition to direct cytotoxic affects, intravesical therapies can incite an anti-tumor immune response by recruiting or depleting effector (CD4+, CD8+) and regulatory (Foxp3+) T cells to the tumor microenvironment (Powles T, et al., Urol Int, 79(1):67-72 (2007)). Intravesical treatment with CDDP solution and PAA-CDDP NPs resulted in significantly lower numbers of CD8+ cells (P<0.05) compared to untreated controls, without differences in the number of CD3+ and Foxp3+ cells (FIGS. 11-13). These results indicated that locally delivered CDDP induced an immune response, consistent with known immunologic effects of systemically delivered cisplatin (Boeh H, et al., Ann Oncol, 26(11):2305-2310 (2015); Hjelle L v, et al., J Clin Oncol Conf, 32:15 SUPPL. 1 (2014)).

The results showed that more than 1 μg/ml of cisplatin was detected in plasma within 1 h after intravesical administration of CDDP solution to mice, consistent with the hypersensitivity observed with intravesical CDDP therapy in humans. This level of serum platinum is notable when considering that platinum concentrations as low as 0.23 ng/ml have been documented years after systemic treatment and have been linked to oto- and neuro-toxicities, as well as hypogonadism, hypercholesterolemia, and hypertension (Boeh H, et al., Ann Oncol, 26(11):2305-2310 (2015); Hjelle L v, et al., J Clin Oncol Conf, 32:15 SUPPL. 1 (2014)).

Although studies have shown PEG_{high}-PAA-CDDP NPs were more efficacious for local treatment of lung cancer and glioblastoma than PAA-CDDP NPs in orthotopic murine models (Paraskar A S, et al., Proc Natl Acad Sci USA, 107(28):12435-12440; Sengupta P, et al., Proc Natl Acad Sci, 109(28):11294-11299), PEGylation did not turn out to be beneficial for local treatment of early-stage bladder cancer. Increasing the amount of PEG seemed to have a negative impact on CDDP uptake and retention in bladder tissue. CDDP levels in mouse bladder tissue 1 h after intravesical administration were substantially lower after administration of PEG_{high}-PAA-CDDP NPs compared to PAA-CDDP NPs. Moreover, only mice treated with PAA-CDDP NPs had detectable levels of CDDP in their bladder tissue 4 h after intravesical administration. Similarly, increasing PEG content led to decreased CDDP levels in bladder tissue 1 h after intravesical administration in rats, and only rats treated with PAA-CDDP NPs had detectable CDDP levels in bladder tissue 4 h after administration. Due to the assembly nature of the PAA-CDDP complex formation, PEGylated NP formulations differed in size and CDDP content compared to PAA-CDDP NPs.

The data showing improved in vivo efficacy (e.g., significantly lower Ki67+ cells) in the PAA-CDDP NPs treated formulations compared to untreated tumors indicated it was a promising alternative to conventional CDDP. Additionally, no rats treated with PAA-CDDP NPs had evidence of invasion into the lamina propria, which is notable because patients with lamina propria invasion (T1) have a worse prognosis (5 yr cancer-specific survival 88%) than patients with carcinoma in situ (CIS) or high grade papillary disease (5 yr CSS 98%) (Knowles M A, et al, Nat Publ Gr, 15(1):25-41 (2015)). Additionally, T1 invasion is a significant risk factor for stage progression (to muscle invasive bladder cancer (MIBC)), which is also associated with a worse cancer prognosis (CSS 63%). Furthermore, muscle invasion often necessitates chemoradiation or bladder extirpation and urinary reconstruction, both of which are associated with significant morbidity. As a result, preventing invasive disease is an important endpoint and is a major reason for pre-clinical studies to be performed in clinically relevant NMIBC animal models.

In summary, CDDP NPs reduced side effects with intravesical use by limiting systemic exposure and local toxicity, while maintaining CDDP's primary anti-tumor efficacy in vitro and in vivo. That is, nanoparticle encapsulation of cisplatin led to decreased systemic drug exposure, decreased local toxicity, increased bladder tissue drug concentrations, and improved efficacy against bladder cancer. Counterintuitively, increasing amounts of PEG on the particle surface led to decreased delivery to bladder tissue, and decreased efficacy in the cancer model. Of note, the bladder epithelium has distinct features compared to other mucosal epithelia, including a lack of a secreted mucus layer.

### Example 4: Docetaxel (DTX) Nanosuspension/Nanocrystal Retained Cytotoxicity Against Tumor Cells In Vitro

FIG. 14 shows the sizes and their polydispersity of DTX nanosuspensions formed with PLURONIC® F127, PLURONIC® F68, KOLLIPHOR® HS 15, or KOLLIPHOR® TPGS.

FIG. 15 shows the sizes and polydispersity of DTX nanosuspension over time.

Table 3 illustrates the physical parameters of formed DTX nanosuspension.

TABLE 3
Parameters of an exemplary DTX nanosuspension.

| Particle Size | Polydispersity Index | Zeta potential |
|---|---|---|
| 230 ± 5 nm | 0.15 ± 0.01 | −1.6 ± 0.1 mV |

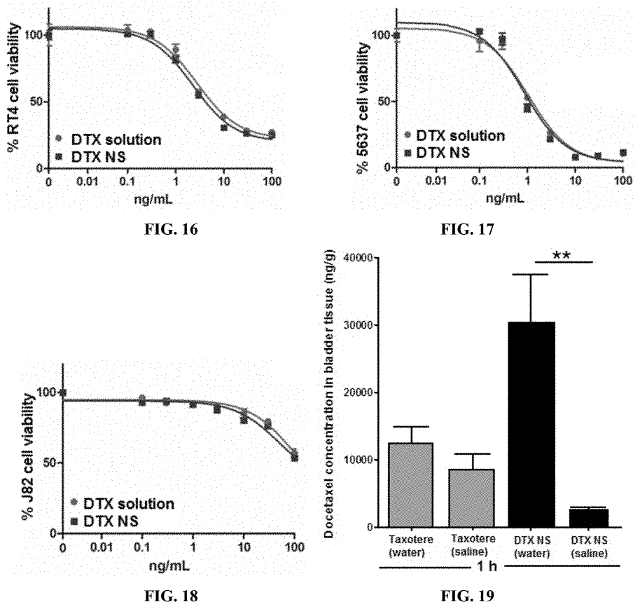

FIGS. 16-18 show DTX nanosuspension (DTX NS) retained the anti-cancer activity of DTX, where the IC₅₀ against each of RT4, 5637, and J82 cells was similar to that of free DTX in solution.

### Example 5: In Vivo Efficacy of Enhanced Drug Retention in Bladder, Low Systemic Amount, and Improved Prevention of Bladder Cancer Progression of DTX Nanosuspension Delivered in Water

Docetaxel nanocrystals (or nanosuspensions) were compared to a by instillation into voided bladder at a dose of 0.7 mg/mL of docetaxel in either water or saline to rats. FIG. 19 shows DTX NS delivered in water resulted in a significantly greater amount of DTX concentration in bladder tissue than DTX NS delivered in saline at 1 hour time point post administration. DTX NS delivered in water also led to a greater amount of DTX concentration in bladder tissue than free Taxotere delivered in either water or saline. Taxotere delivered in water and in saline resulted in a similar amount of docetaxel in bladder, both lower than DTX nanosuspension delivered in water.

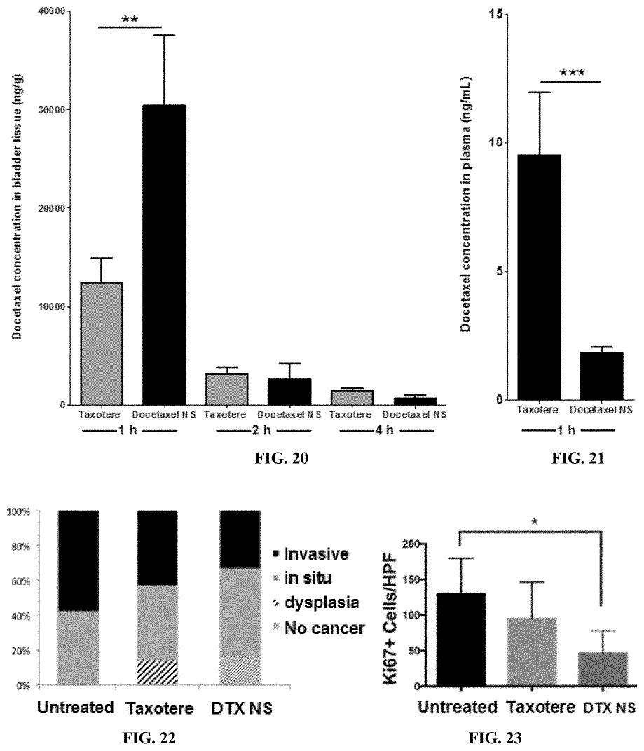

FIG. 20 shows the follow-up time points of uptake and retention of docetaxel in rats after intravesical administration, where administration with DTX NS formulation in water greatly enhanced the amount of docetaxel in bladder at 1 hour than Taxotere formulation in water. clinical soluble version of docetaxel, Taxotere.

### 1. DTX Nanosuspension Delivered in Water Increased Bladder Tissue Uptake Compared to Delivery in Saline, and was Superior to Delivery in the Soluble Taxotere Form.

Docetaxel nanosuspension (DTX NS) and Taxotere (0.3 mL) were administered

FIG. 21 shows the plasma level of docetaxel at 1 hour post intravesically administration in the form of DTX NS was significantly lower than that delivered in the soluble form, Taxotere. The plasma levels of docetaxel for both Taxotere and DTX NS formulation at 2-hour and 4-hour time points were below the limit of quantification, 1 ng/mL.

### 2. DTX Nanosuspension Prevented Bladder Tumor Progression.

In a MNU-induced rat bladder carcinogenesis model as described in Example 3, Fischer Rat received weekly intravesical administration of Taxotere, DTX NS, or saline, for six weeks.

FIGS. 22 and 23 show DTX NS prevented progression of bladder tumors, which was significantly improved over untreated (saline) group, whereas Taxotere formulation did not show a significant improvement over untreated group. Docetaxel nanosuspension was more effective than taxotere suspension in preventing transition of in situ bladder cancer to invasive cancer type (FIG. 22). Decreased Ki67+ staining confirmed lack of proliferating cells (cancer) in the bladders of mice treated with nanosuspension (FIG. 23).

We claim:

1. A hypotonic formulation comprising
particles comprising a biocompatible polymer and a therapeutic, prophylactic, diagnostic or nutraceutical agent,
in an medium hypotonic for urothelium,
such that the formulation causes the particles to transport through the urothelial epithelium where the particles dissemble or dissolve to release the agent.

2. The formulation of claim 1, wherein the particles in the medium are in a form comprising micelles, colloids, liposomes, vesicles, nanodroplets, nano-structured hydrogel, nanocrystals, or nanosuspension.

3. The formulation of claim 1, wherein the polymer does not comprise polyethylene oxide or comprises no more than 50% polyethylene oxide by weight.

4. The formulation of claim 1, wherein the urothelial epithelium comprises epithelium of renal pelvis, ureters, bladder, or urethra.

5. The formulation of claim 1, wherein the medium is water.

6. The formulation of claim 1, wherein the medium comprises a pharmaceutical excipient less than 220 mOsm/kg.

7. The formulation of claim 1, wherein the polymer comprises polyamino acids or poloxamers.

8. The formulation of claim 1 wherein the agent is selected from the group consisting of analgesics, anti-inflammatory drugs, anti-proliferatives, immunotherapeutic agents, anti-infectious agents, antihistamines, corticosteroids, dopaminergics, and muscle relaxants.

9. The formulation of claim 8, wherein the agent is an anti-proliferative selected from the group consisting of cisplatin, transplatin, carboplatin, dicycloplatin, oxaliplatin, picoplatin, aroplatin, gemcitabine, cabazitaxel, docetaxel, doxorubicin, mitoxantrone, bleomycin, daunorubicin, dactinomycin, epirubicin, idarubicin, mitomycin, pentostatin, epirubicin, valrubicin, and combinations thereof.

10. The formulation of claim 9 wherein the agent is gemcitabine alone or in combination with other chemotherapeutic agents.

11. The formulation of claim 10 wherein the agent is the combination of cabazitaxel, cisplatin and gemcitabine.

12. The formulation of claim 9 wherein the agent is an immunotherapeutic selected from the group consisting of interferons and Atezolizumab (Tecentriq).

13. The formulation of claim 1, wherein the polymer is bonded with the agent.

14. The formulation of claim 1, wherein the agent is encapsulated in the particles.

15. The formulation of claim 1, wherein the particles are nanoparticles.

16. A method of making a hypotonic formulation comprising
suspending particles comprising a biocompatible polymer and a therapeutic, prophylactic, diagnostic or nutraceutical agent, in an medium hypotonic for urothelium, such that the formulation causes the particles to transport through the urothelial epithelium where the particles dissemble or dissolve to release the agent.

17. A method of administering one or more therapeutic, prophylactic, and/or diagnostic agents to a subject in need thereof, comprising administering to the bladder an effective amount of the formulation of claim 1.

18. The method of claim 17, wherein the formulation is administered to the bladder via intravesical instillation.

19. The method of claim 17, wherein the subject has a bladder infection.

20. The method of claim 17 wherein the subject has bladder cancer.

21. The method of claim 17 resulting in a reduced concentration of the agent in plasma compared to administering of the agent without the particles.
